(19) United States
(12) Patent Application Publication
(10) Pub. No.: US 2026/0157977 A1
(43) Pub. Date: Jun. 11, 2026

(54) OCCLUSIVE PLASTER WITH FLEXIBLE BACKING

(71) Applicant: LTS Lohmann Therapie-Systeme AG, Andernach (DE)

(72) Inventors: Horst DZEKAN, Meinborn (DE); Nico REUM, Mendig (DE); Marco EMGENBROICH, Rheinbach (DE); Luisa Wittlich, Freiburg im Breisgau (DE)

(21) Appl. No.: 18/707,337

(22) PCT Filed: Nov. 4, 2022
(86) PCT No.: PCT/EP2022/080882

§ 371 Date: May 3, 2024

(30) Foreign Application Priority Data
Nov. 5, 2021 (DE) 10 2021 128 912.2

(51) Int. Cl. A61K 9/70; A61K 31/381; A61K 47/02; A61K 47/10; A61K 47/22; A61K 47/32

(52) U.S. Cl. CPC A61K 9/7061; A61K 9/7069; A61K 31/381; A61K 47/02; A61K 47/10; A61K 47/22; A61K 47/32

(57) ABSTRACT

The invention relates to a transdermal therapeutic system comprising a backing layer, an occlusive layer and at least one pharmaceutically active ingredient, the occlusive layer including at least one occlusive adhesive component on the basis of at least one polyisobutylene and at least one styrene block copolymer, and such a system for use as a medicinal product.

The invention relates to a transdermal therapeutic system and its use as a medicinal product.

Transdermal therapeutic systems (TTS) are dosage forms for the administration of drugs in form of a patch. These systems have certain advantages over conventional dosage forms. When the patch containing the active ingredient is applied on the skin, said active ingredient will be absorbed through the skin of the corresponding part of the body exactly in this area and with an exact dose, and without premature degradation in the gastrointestinal tract or liver. In addition, this dosage form enables a constant release of the active ingredient over a longer period of time.

In many cases, the transdermal administration of active ingredients is hampered by the low permeability of the skin. It was therefore important to increase the permeability of the skin for an efficient absorption of active ingredients. One possibility for this is the effect of occlusion, which is understood to mean a covering or sealing of a skin area to the maximum extent possible causing an accumulation of water vapor in the upper layers of the skin, which, in turn, causes a higher permeability of the skin in relation to the active ingredient.

However, these well-known patches have the drawback, that their material properties generally are rather inelastic and rigid, which makes them less comfortable to wear, thus limiting the wearer's mobility and causing the patch to come off frequently and unintentionally.

According to DE 10103860 A1, the occlusion of the patch is achieved by using a backing layer that is impermeable to water vapor, such as a thin plastic film, preferably a polyethylene terephthalate (PET) film.

Another possibility to increase the permeability of the skin for the absorption of an active ingredient is the use of penetration enhancers.

For example, EP 1 312 360 A1 discloses an analgesic, anti-inflammatory patch (“Dojin Patch”) for the local release of diclofenac. The system includes N-methyl-2-pyrrolidone as a solvent and thus ensures increased permeation of the active ingredient through the skin. The drawback of this system is that this solvent is a substance of concern for human health, and according to ICH guideline Q3C (dated Feb. 4, 2011), a daily intake of 5.3 mg N-methyl-2-pyrrolidone should not be exceeded.

The object of the present invention is to remove the above-mentioned drawbacks of the prior art. More particularly, the object of the present invention is to provide a transdermal therapeutic system which produces an optimal absorption of an active ingredient via the skin, and at the same time does not require any solvents which are of concern for the health, i.e. the optimal absorption of an active ingredient via the skin should preferably only be brought about via occlusion. Despite the occlusion, the system should still offer a high level of comfort and adhesion, even on flexible parts of the body such as joints.

The above object is solved by a transdermal therapeutic system according to claim 1, i.e. by a transdermal therapeutic system comprising a backing layer, an occlusive layer and at least one active pharmaceutical ingredient, wherein the occlusive layer comprises at least one occlusive adhesive component on the basis of at least one polyisobutylene and at least one styrene block copolymer.

When describing the transdermal therapeutic system according to the invention, the term “comprising” may also mean “consisting of”.

In one embodiment, the transdermal therapeutic system according to the invention may comprise, as far as the layer structure is concerned, the backing layer and the occlusive layer. In this embodiment, the at least one active pharmaceutical ingredient is contained in the occlusive layer.

In another embodiment, the transdermal therapeutic system according to the invention may comprise, in addition to the backing layer and the occlusive layer, further layers which are preferably arranged such that the occlusive layer is arranged between the backing layer and the respective further layer. Such additional layers are often referred to as matrix layers. If the transdermal therapeutic system according to the invention comprises such a matrix layer in addition to the backing layer and the occlusive layer, the at least one active pharmaceutical ingredient is contained in the matrix layer.

Occlusion refers to the covering or sealing of skin regions, at least to the maximum extent possible, with materials that are impermeable to water vapor. As a result, the insensible perspiration (perspiratio insensibilis, water or water vapor release through the skin of a person at rest) is impaired, leading to an accumulation of moisture, and consequently to hydration of the stratum corneum (outermost layer of the epidermis). Under occlusive conditions, the water content of the stratum corneum increases by up to 25% (m/m), preferably by up to 50% (m/m). The surface temperature of the skin can also rise to 37° C. Preferably, the amount of water vapor released is less than 500 g/m², particularly preferably less than 200 g/m², and most preferably less than 120 g/m² within 24 h, measured according to DIN EN 13726-2:2002 at a temperature of 37° C. and a relative humidity of 30%.

An adhesive component is understood to be a substance that is either an adhesive in itself, preferably a pressure-sensitive adhesive, i.e. is tacky in itself, or produces an adhesive when mixed with other substances. An adhesive is a substance which, as defined in DIN EN 923 (June 2008), is a non-metallic substance capable of joining parts by surface bonding (adhesion) and adequate internal strength (cohesion). A substance or mixture of substances is described as tacky if it can be used as an adhesive in itself, more particularly as a pressure-sensitive adhesive. Pressure-sensitive adhesives are adhesives that remain highly viscous and permanently tacky after being applied to a carrier material and can then be applied to a substrate by applying light pressure and remain stuck. Pressure-sensitive adhesives, as defined in DIN EN 923 (June 2008), are also characterized by the fact that their set, dry film is permanently tacky at room temperature, as defined in DIN EN 923 (June 2008), and remains adhesive. Adhesive bonds produced with pressure-sensitive adhesives can usually be removed without destroying the bonded substrates.

The at least one occlusive adhesive component preferably comprises a pressure-sensitive adhesive or gives a pressure-sensitive adhesive by mixing with other substances.

The at least one occlusive adhesive component is thus to be understood to be a component that largely prevents insensible perspiration, i.e. the loss of water vapor from the skin, and thus leads to an accumulation of moisture in the stratum corneum. Preferably, the at least one occlusive adhesive component is an occlusive adhesive component that is already inherently tacky.

Permeability is the ability of solids (including porous solids), especially thin partitions, to let pass certain substances (gases, liquids, dissolved molecules, ions, or atoms). In the present case, the ability of human or animal skin to let pass small molecules, more particularly active pharmaceutical ingredients. In technical terms, permeation is understood to be the process of one substance passing through or penetrating another. The term is often used in the context of the penetration of cosmetic or active pharmaceutical ingredients into or through the skin.

The use of an occlusive adhesive component based on a polyisobutylene therefore increases the permeability of the skin in relation to the active ingredient.

The use of a styrene-isoprene block copolymer has the advantage that the occlusion can be further positively influenced. In addition, the styrene-isoprene block copolymer reinforces the adhesive strength of the polyisobutylene for a better adhesion of the individual layers of the transdermal therapeutic system.

In a preferred embodiment, the backing layer is not occlusive.

In a preferred embodiment, the matrix layer is not occlusive.

In one embodiment, the transdermal therapeutic system according to the invention is preferably characterized in that the at least one polyisobutylene is a mixture comprising a medium molecular weight polyisobutylene and a high molecular weight polyisobutylene.

The medium molecular weight polyisobutylene, as described above, preferably is a polyisobutylene with an average molecular weight (determined from viscosity measurements) of 20,000 to 60,000 g/mol, more particularly about 40,000 g/mol (determined, for example, by gel permeation chromatography).

The medium molecular weight polyisobutylene, as described above, preferably is a polyisobutylene with a limiting viscosity of about 27.5 to 51.6 cm³/g.

An example of a commercially available, suitable medium molecular weight polyisobutylene is available under the trade name Oppanol B10 from BASF.

The high molecular weight polyisobutylene, as described above, preferably is a polyisobutylene with an average molecular weight (determined from viscosity measurements) of 1,000,000 to 1,200,000 g/mol, more particularly about 1,110,000 g/mol (determined, for example, by gel permeation chromatography).

The high molecular weight polyisobutylene, as described above, preferably is a polyisobutylene with a limiting viscosity of about 128 to 479 cm³/g.

An example of a commercially available, suitable high molecular weight polyisobutylene is available under the trade name Oppanol B100, which is also known under the trade name Oppanol N100 from BASF.

In one embodiment, the transdermal therapeutic system according to the invention is preferably characterized in that the at least one styrene block copolymer comprises a styrene-isoprene-styrene block copolymer, a styrene-ethylene-styrene block copolymer, a styrene-butadiene-styrene block copolymer, a styrene-ethylene-butylene-styrene block copolymer, and or a styrene-ethylene-propylene-styrene block copolymer.

Styrene block copolymers are understood to be block copolymers (or segment copolymers) that consist of or comprise long sequences or blocks of styrene monomers and blocks of at least one other monomer (for example -(AAA)ₓ-(BBB)_{y}-, where A=styrene and B=other monomer).

Styrene block copolymers are also known as styrol block copolymers.

A styrene-isoprene-styrene (SIS) block copolymer is preferred. One of such copolymers preferably comprises or consists of long sequences or blocks of styrene monomers, followed by long sequences or blocks of isoprene monomers, again followed by long sequences or blocks of styrene monomers (for example -(AAA)ₓ-(BBB)_{y}-(AAA)_{z}-, where A=styrene and B=isoprene).

In one embodiment, the transdermal therapeutic system according to the invention is preferably characterized in that the at least one polyisobutylene is a mixture comprising a medium molecular weight polyisobutylene and a high molecular weight polyisobutylene in a weight ratio of 95:5 to 75:25.

Preferred are mixtures comprising a medium molecular weight polyisobutylene and a high molecular weight polyisobutylene, more particularly as defined above, in a weight ratio of 95:5, 94:6, 93:7, 92:8, 91:9, 90:10, 89:11, 88:12, 87:13, 86:14, 85:15, 84:16, 83:17, 82:18, 81:19, 80:20, 79:21, 78:22, 77:23, 76:24, or 75:25. Mixtures comprising a medium molecular weight polyisobutylene and a high molecular weight polyisobutylene, more particularly as defined above, in a weight ratio of 96:4, 97:3, 98:2, 99:1, 74:26, 73:27, 72:28, 71:19, or 70:30 are possible as well.

In one embodiment, the transdermal therapeutic system according to the invention is preferably characterized in that the at least one polyisobutylene is contained in the occlusive layer in an amount of 80 to 97.5 wt %, preferably 85 to 95 wt %, based on the total weight of the occlusive layer.

In one embodiment, the transdermal therapeutic system according to the invention is preferably characterized in that the medium molecular weight polyisobutylene is contained in the occlusive layer in an amount of 55 to 90 wt %, preferably of 60 to 85 wt %, more preferably if 65 to 80%, based on the total weight of the occlusive layer.

In one embodiment, the transdermal therapeutic system according to the invention is preferably characterized in that the high molecular weight polyisobutylene is contained in the occlusive layer in an amount of 5 to 35 wt %, preferably of 10 to 30 wt %, more preferably if 15 to 25 wt %, based on the total weight of the occlusive layer.

If a mixture of different polyisobutylene is used, this quantity refers to the total amount of polyisobutylene.

In one embodiment, the transdermal therapeutic system according to the invention is preferably characterized in that the at least one styrene block copolymer is contained in the occlusive layer in an amount of 2.5 to 20 wt %, preferably of 5 to 15 wt %, based on the total weight of the occlusive layer.

In one embodiment, the transdermal therapeutic system according to the invention is preferably characterized in that the basis weight of the occlusive layer is from 30 to 200 g/m², preferably from 50 to 150 g/m².

A lower basis weight is disadvantageous, since the occlusion cannot be established to such an extent that an acceptable water vapor permeability can be reached.

With higher basis weights, the water vapor permeability is acceptable, but the transdermal therapeutic systems become less flexible and less economical due to the increased use of material.

In one embodiment, the transdermal therapeutic system according to the invention is preferably characterized in that the water vapor permeability of the occlusive layer is less than 120 g/m², preferably less than 100 g/m² within 24 hours.

The water vapor permeability is determined here according to DIN EN 13726-2:2002 at a temperature of 37° C. and a relative humidity of 30%.

In one embodiment, the transdermal therapeutic system according to the invention is preferably characterized in that the transdermal therapeutic system additionally comprises a matrix layer, wherein the occlusive layer is arranged between the backing layer and the matrix layer, wherein the matrix layer comprises at least one pressure-sensitive adhesive and the at least one active pharmaceutical ingredient.

The at least one active pharmaceutical ingredient may be present in the matrix layer only, or in both the matrix layer and the occlusive layer.

In another embodiment, a matrix layer is present, but the at least one active pharmaceutical ingredient is nevertheless present exclusively in the occlusive layer. The matrix layer is then preferably used to strengthen the adhesion.

In one embodiment, the transdermal therapeutic system according to the invention is preferably characterized in that the at least one pressure-sensitive adhesive comprises a pressure-sensitive adhesive on the basis of silicone, on the basis of a (meth)acrylate polymer and/or on the basis of a (meth)acrylate copolymer.

(Meth)acrylate polymers are suitable, more particularly in the form of self-crosslinking (meth)acrylic acid-containing (meth)acrylate polymers, which crosslink by adding aluminum or titanium compounds to form chelate esters. In such self-crosslinked matrix polymers, the (meth)acrylic acid bound to, e.g., the titanium forms crosslinking points. Alternatively, non-self-crosslinking (meth)acrylate copolymers, for example those with hydroxyl or acid groups as functional groups, can be used. Such polymers are commercially available, e.g., under the trade name Durotak from Henkel.

Particularly suitable acrylate polymers that can be mentioned are copolymers or terpolymers of 2-ethylhexyl acrylate, vinyl acetate, 2-hydroxyethyl acrylate; copolymers or terpolymers of 2-ethylhexyl acrylate, vinyl acetate and acrylic acid; copolymers or tetrapolymers of 2-ethylhexyl acrylate, butyl acrylate, vinyl acetate and acrylic acid, or a mixture thereof.

Most particularly suitable are self-crosslinking tetrapolymers of 2-ethylhexyl acrylate, butyl acrylate, vinyl acetate and acrylic acid and self-crosslinking or non-self-crosslinking terpolymers of 2-ethylhexyl acrylate, vinyl acetate and 2-hydroxylethyl acrylate.

Special acrylate polymers are, for example:
Duro-Tak™ 387-2510 or Duro-Tak™ 87-2510 (a copolymer based on 2-ethylhexyl acrylate, 2-hydroxyethyl acrylate, and methyl acrylate),
Duro-Tak™ 87-4287 (a copolymer based on vinyl acetate, 2-ethylhexyl acrylate and 2-hydroxyethyl acrylate as a solution in ethyl acetate without crosslinker),
Duro-Tak™ 387-2287 or Duro-Tak™ 87-2287 (a copolymer based on vinyl acetate, 2-ethylhexyl acrylate, 2-hydroxyethyl acrylate and glycidyl methacrylate as a solution in ethyl acetate without crosslinker),
Duro-Tak™ 387-2516 or Duro-Tak™ 87-2516 (a copolymer based on vinyl acetate, 2-ethylhexyl acrylate, 2-hydroxyethyl acrylate and glycidyl methacrylate as a solution in ethyl acetate, ethanol, n-heptane, and methanol with a titanium crosslinker),
Duro-Tak™ 387-2051 or Duro-Tak™ 87-2051 (a copolymer based on acrylic acid, butyl acrylate, 2-ethylhexyl acrylate and vinyl acetate as a solution in ethyl acetate and heptane),
Duro-Tak™ 387-2353 or Duro-Tak™ 87-2353 (a copolymer based on acrylic acid, 2-ethylhexyl acrylate, glycidyl methacrylate and methyl acrylate as a solution in ethyl acetate and hexane),
Duro-Tak™ 87-4098 (a copolymer based on 2-ethylhexyl acrylate and vinyl acetate as a solution in ethyl acetate),
Duro-Tak™ 87-900A
Duro-Tak™ 387-2052 (a copolymer based on acrylic acid, butyl acrylate, 2-ethylhexyl acrylate and vinyl acetate as a solution in ethyl acetate, ethanol, isopropanol, and heptane with an aluminum crosslinker),
Duro-Tak™ 87-9301
Duro-Tak™ 87-2074 (a copolymer based on acrylic acid, methyl methacrylate, 2-ethylhexyl acrylate, 2-hydroxyethyl acrylate and glycidyl methacrylate as a solution in ethyl acetate, ethanol, n-heptane, and methanol with an aluminum crosslinker)
Duro-Tak™ 87-235A
Gelva GMS 9073
Gelva GMS 788

In one possible embodiment, the pressure-sensitive adhesive comprises a silicone-based pressure-sensitive adhesive, i.e. a silicone pressure-sensitive adhesive, more particularly an amine-resistant silicone pressure-sensitive adhesive.

Preferably, the silicone pressure-sensitive adhesives that can be used according to the invention are pressure-sensitive adhesives based on silicone polymers, such as a dimethiconol/trimethylsiloxysilicate crosspolymer and/or a trimethylsilyl-treated dimethiconol/trimethylsiloxysilicate crosspolymer, which preferably contain at least 30 wt %, more particularly 35 to 95 wt %, particularly preferably 40 to 90 wt %, or 40 to 60 wt %, or 45 to 55 wt % of silicone polymer(s), based on the silicate.

In one embodiment, the silicone pressure-sensitive adhesives which can be used according to the invention are pressure-sensitive adhesives which are prepared on the basis of silicone polymers, such as dimethiconol/trimethylsiloxysilicate crosspolymers and/or a trimethylsilyl-treated dimethiconol/trimethylsiloxysilicate crosspolymers, which preferably contain 40 wt % of silicone polymers and 60 wt % of silicate. Such an adhesive can be described as a “medium tack adhesive”.

In another embodiment, the silicone pressure-sensitive adhesives which can be used according to the invention are pressure-sensitive adhesives which are prepared on the basis of silicone polymers, such as dimethiconol/trimethylsiloxysilicate crosspolymers and/or a trimethylsilyl-treated dimethiconol/trimethylsiloxysilicate crosspolymer, which preferably contains 45 wt % of silicone polymers and 55 wt % of silicate. Such an adhesive can be described as “high-tack-adhesive”.

Mixtures of different silicone adhesives can also be used, for example a 1:1 (by weight) ratio of a “medium tack adhesive” and a “high tack adhesive” as described above.

All silicone pressure-sensitive adhesives, as described above, preferably contain n-heptane as a solvent.

Suitable silicone pressure-sensitive adhesives for use according to the present invention are, e.g., the hot-melt pressure-sensitive adhesives BIO-PSA 7-4201 and/or BIO-PSA 7-4301 from Dow Corning. BIO-PSA 7-4201 is a “medium tack adhesive” and BIO-PSA 7-4301 is a “high tack adhesive”, as defined above.

It is also possible to use mixtures of the aforementioned polymers as pressure-sensitive adhesives, with the proviso that the polymers are sufficiently compatible with each other so that no substantial segregation of the polymer components occurs. However, due to the higher processing effort required for the production of pressure-sensitive adhesive layers based on different polymers, it is preferable if the transdermal therapeutic system contains only one type of polymer as pressure-sensitive adhesive.

The transdermal therapeutic system according to the invention is preferably characterized in that the at least one pressure-sensitive adhesive is present in the matrix layer in an amount of 40 to 98 wt %, preferably 50 to 95 wt %, particularly preferably 60 to 90 wt %, based on the matrix layer.

In one embodiment, the transdermal therapeutic system according to the invention is preferably characterized in that the at least one active pharmaceutical ingredient is selected from the group comprising hypnotics, sedatives, antieleptics, amphetamines, psychoneurotropics, neuroleptics, neuromuscular-blocking agents, antispasmodics, antihistamines, antiallergics, cardiotonic agents, antiarrhythmics, diuretics, hypotensive agents, vasopressors, antitussives, expectorants, analgesics, thyroid hormones, sex hormones, glucocorticoid hormones, antidiabetics, antitumor agents, antibiotics, chemotherapeutics, narcotics, anti-Parkinson agents, anti-Alzheimer agents and/or triptans, wherein this group is not exhaustive.

Specific examples comprise acetaminophen, adrenaline, agomelatine, alprazolam, amlodipine, anastrozole, apomorphine, aripiprazole, asenapine, atorvastatin, baclofen, benzocaine, benzocaine/menthol, benzydamine, buprenorphine, buprenorphine/naloxone, buprenorphine/naloxone/ketirizine, cannabinoids, capsicum, cetirizine, chlorpheniramine, clomipramine, dexamethasone, dextromethorphan, dextromethorphan/phenylephrine, diclofenac, diphenhydramine, diphenhydramine/phenylephrine, donepezil, dronabinol, epinephrine, escitalopram, estradiol, estradiol/levonorgestrel, ethinylestradiol, famotidine, fentanyl, fingolimod, glimepiride, GLP-1 peptides, granisetron, ibuprofen, insulin, insulin nanoparticles, insulin/GLP-1 nanoparticles, ketamine, ketoprofen, ketotifen, caffeine, levocetirizine, levonorgestrel, lidocaine, loperamide, loratadine, loxoprofen, meclizine, methylphenidate, methyl salicylate, midazolam, mirodenafil, montelukast, multimeric-001, naloxone, nicotine, nitroglycerin, norelgestromin, olanzapine, olopatadine, ondansetron, oxybutynin, pectin, pectin/menthol, pectin/ascorbyl acid, pediaSUNAT (artesunate and amodiaquine), piroxicam, phenylephrine, prednisolone, pseudoephedrine, risperidone, rivastigmine, rizatriptan, rotigotine, salbutamol, selegiline, senna glycosides, sildenafil citrate, simethicone, xumatriptan, tadalafil, testosterone, triamcinolone acetonide, triptan, tropicamide, voglibose, zolmitriptan, zolpidem, or pharmaceutically acceptable salts of these compounds.

The active pharmaceutical ingredient can also be a mixture of different active ingredients.

In one embodiment, the transdermal therapeutic system according to the invention is preferably characterized in that the at least one active pharmaceutical ingredient is present in an amount of from 0.5 to 40 wt %, preferably from 1 to 30 wt % in the occlusive layer, based on the total weight of the occlusive layer, and/or in the matrix layer, based on the total weight of the matrix layer.

In one embodiment, the transdermal therapeutic system according to the invention is preferably characterized in that the matrix layer and/or the occlusive layer comprises at least one penetration enhancer, preferably selected from alcohols, fatty acids and/or fatty acid esters.

A penetration enhancer is understood to be a compound that enhances the permeation of one substance through another one, i.e. increases the permeation rate or generally increases the efficiency of permeation.

In addition, the at least one penetration enhancer is preferably a compound which preferably stabilizes the active ingredient form and ensures a relatively high and also stable absorption of the active ingredient via the skin over a longer period of time.

Various mechanisms for increasing penetration are known, such as a lowering of the melting point of the active pharmaceutical ingredient and/or the reduction of the skin barrier, e.g. by opening tight junctions in the skin.

Penetration enhancers are preferably characterized by at least one of the following properties.

Ideally, the effect of penetration enhancers is fast, and the activity and duration of the effect should be both predictable and reproducible.

Penetration enhancers should not have any pharmacological activity within the body, i.e. they should not bind to any receptor sites, for example.

Penetration enhancers preferably should function unidirectionally, i.e. they should allow therapeutic active ingredients to penetrate the body and at the same time prevent the loss of endogenous material from the body.

When penetration enhancers are removed from the skin, the barrier properties should return both quickly and completely.

Penetration enhancers should be suitable for formulation in different formulations, i.e. they should be compatible with both excipients and drugs.

Penetration enhancers should be cosmetically acceptable and feel good on the skin, be non-toxic, non-irritating and non-allergenic.

The function and properties of penetration enhancers are described, for example, in the publication by Williams et al. “Penetration Enhancers”, Advanced Drug Delivery reviews, 56 (2004), 603 to 618, or in the publication by Amjadi et al. “Recent advances in skin penetration enhancers for transdermal gene and drug delivery”, Current Gene Therapy 17 (2), 2017, or in the publication by Gupta et al. “Effect of chemical penetration enhancers on skin permeability: In silico screening using molecular dynamics simulations”, Scientific Reports, 9_1456 (2019), the contents of which are hereby incorporated in full.

Suitable penetration enhancers comprise fatty acids and/or fatty acid esters, such as pentanoic acid, hexanoic acid, octanoic acid, nonanoic acid, decanoic acid, lauric acid, myristic acid, palmitic acid, stearic acid, arachidic acid, behenic acid, lignoceric acid, isoverlinic acid, neoheptonic acid, neonanonic acid, isostearic acid, oleic acid, palmitoleic acid, linolenic acid, vaccenic acid, petroselinic acid, elaidic acid, oleic acid, arachidonic acid, gadoleic acid, erucic acid, ethyl acetate, methyl propylate, butyl acetate, methyl valerate, diethyl sebacitate, methyl laurate, ethyl oleate, isopropyl decanoate, isopropyl myristate (myristic acid isopropyl ester), isopropyl palmitate and/or isopropyl oleate.

Other suitable penetration enhancers comprise polyhydric alcohols, such as propylene glycol or dipropylene glycol.

Other suitable penetration enhancers comprise propylene urea, dimethyl propylene urea and/or dimethyl ethylene urea.

The transdermal therapeutic system according to the invention is preferably characterized in that the occlusive layer and/or the matrix layer contain at least one penetration enhancer in an amount of 0.5 to 20 wt %, particularly preferably in an amount of 2 to 15 wt % and most particularly preferably in an amount of 5 to 10 wt % in the occlusive layer, based on the total weight of the occlusive layer, and/or in the matrix layer, based on the total weight of the matrix layer.

Furthermore, the transdermal therapeutic system according to the invention is characterized in that no penetration enhancers from the class of pyrrolidones, more particularly N-methyl-2-pyrrolidone, sulfoxides, more particularly dimethyl sulfoxide (DMSO), formamides, more particularly dimethylformamide (DMF), and/or 1-dodecylazacycloheptan-2-one or laurocapram (azone) and/or derivatives are present in the transdermal therapeutic system according to the invention.

In another preferred embodiment, the transdermal therapeutic system according to the invention contains at least one antioxidant, preferably in the matrix layer and/or in the occlusive layer. The at least one antioxidant is preferably selected from alpha-tocopherol, ascorbyl palmitate, sodium metabisulfite (Na₂S₂O₅) and butylhydroxytoluene.

The at least one antioxidant is preferably present in an amount of 0.05 to 1.5 wt %, preferably 0.2 to 1 wt % in the occlusive layer, based on the total weight of the occlusive layer, and/or in the matrix layer, based on the total weight of the matrix layer.

The use of an antioxidant has the advantage that the transdermal therapeutic system according to the invention remains stable over a longer period of time and under various external conditions.

The transdermal therapeutic system according to the invention is further preferably characterized in that the matrix layer has a basis weight of 50 to 400 g/m², preferably 70 to 150 g/m² and particularly preferably 90 to 120 g/m².

In another preferred embodiment, the transdermal therapeutic system according to the invention is characterized in that it comprises a surface area of about 2 to 250 cm², preferably of about 50 to 150 cm².

The transdermal therapeutic system according to the invention is preferably characterized in that the backing layer comprises an elastic woven, knitted, or non-woven fabric.

Said fabric is preferably stretchable in at least one direction, preferably in two directions. This refers to the stretchability or elasticity in the longitudinal and/or transverse direction, but not in the thickness direction, of the woven, knitted, or non-woven fabric.

Elasticity or being stretchable in at least one, preferably in two (longitudinal and/or transverse) directions, is understood to mean the ability of the transdermal therapeutic system to stretch in at least one, preferably in two different directions, preferably in the longitudinal and transverse directions, but not in the thickness direction, in relation to the initial state of the material, without the original shape being lost. Permanent deformation of the stretched material does not occur. Elasticity is evaluated by means of elongation, which is specified as a dimensionless number or, when multiplied by 100, as a percentage value.

The transdermal therapeutic system according to the invention is preferably characterized in that the backing layer has an elasticity of 1 to 100%, preferably of 10 to 50% and most preferably of 15 to 30%, in the longitudinal and/or transverse direction, but not in the thickness direction, in relation to the initial state of the material. Elasticity is determined in accordance with ISO 13934-1 of Apr. 10, 2013.

The use of such a stretchable woven, knitted, or non-woven fabric has the advantage that the transdermal therapeutic system according to the invention or the active ingredient-containing patch, even in large embodiments and when applied to flexible regions of the body, such as joints of the extremities, is very comfortable to wear, does not restrict mobility, and has a high adhesion to the skin, and thus prevents unintentional detachment.

Furthermore, in another preferred embodiment, the transdermal therapeutic system according to the invention is characterized in that it comprises a removable protective layer, preferably of a siliconized polyethylene terephthalate film, which is adhered to the side of the matrix layer which is not the occlusive layer. This removable protective layer (by siliconization or fluoropolymerization (in the case of silicone adhesives) on the side with which the protective layer is in contact with the matrix) makes packaging and transport of the transdermal therapeutic system according to the invention easier.

The present invention further relates to a transdermal therapeutic system as described above for use as a medicinal product.

The invention is explained below with reference to non-limiting examples.

### EXAMPLES

### Example 1

TABLE 1
Composition of the occlusive layers

| | LTS | Composition [%] | | | |
|---|---|---|---|---|---|
| | formulation | Oppanol | Oppanol | SIS | Hydrobrite |
| | code | B10 | N100 | 5229P | HV |
| V1 | 14xGru0016-1 | 85 | 15 | — | — |
| V2 | 14xGru0015-1 | 75 | 25 | — | — |
| V3 | 14xGru0011-1 | Durotak 387-2287 | | | |
| V4 | 14xGru0036- | Durotak 387-2287: 90 | | | 10 |
| | 1n/a | | | | |
| V5 | 14xGru0037- | Durotak 387-2287: 95 | | | 5 |
| | 1n/a | | | | |
| V6 | 14xGru0038- | Durotak 387-2287: 85 | | | 15 |
| | 1n/a | | | | |
| O1 | 14xGru0022 | 80 | 10 | 10 | — |
| O2 | 14xGru0031 | 72.5 | 22.5 | 5 | — |
| O3 | 14xGru0030 | 70 | 20 | 10 | — |
| O4 | 14xGru0034 | 72.5 | 22.5 | — | 5 |
| O5 | 14xGru0023 | 82.5 | 12.5 | 5 | — |
| O6 | 14xGru0024 | 77.5 | 7.5 | 15 | — |
| O7 | 14xGru0032 | 67.5 | 17.5 | 15 | — |
| O8 | n/a | 75 | 15 | 10 | — |
| O9 | n/a | 77.5 | 17.5 | 5 | — |
| O10 | n/a | 72.5 | 12.5 | 15 | — |

### Preparation of the Occlusive Layers:

First, pre-solutions are prepared. The SIS is dissolved in n-propyl acetate so that the solids content is approximately between 20% and 25%. The Oppanols are dissolved in n-heptane. For B10, a solids content is adjusted to be between 60% and 70%, for N100 a solids content of between 15% and 20%. The solutions are mixed together in the correct ratio and homogenized. The finished mixture is spread onto a siliconized release liner in the desired layer thickness, and the laminate is dried in the oven to remove the solvents. Finally, the non-occlusive backing (woven or non-woven) is laminated onto the dried and cooled laminates. The water vapor permeability of various occlusive layers was measured. The water vapor permeability was determined according to DIN EN 13726-2:2002 at a temperature of 37° C. and a relative humidity of 30%.

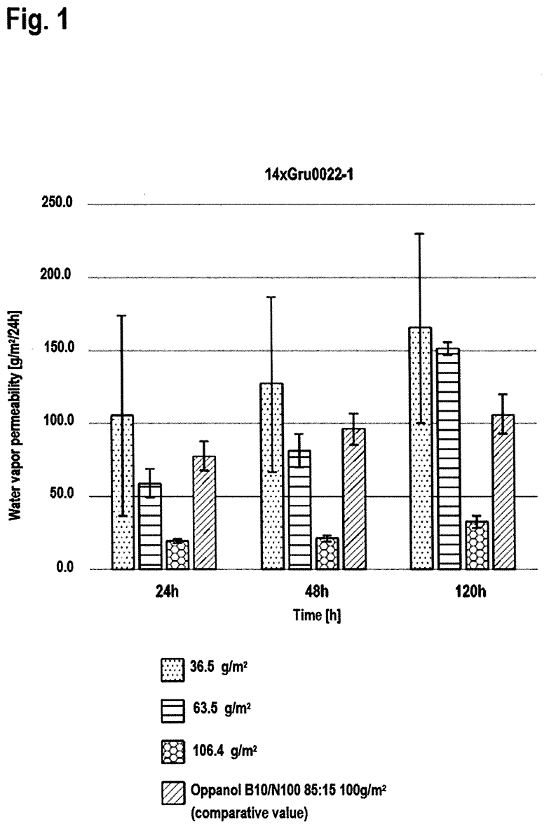

FIG. 1: Water vapor permeability of three occlusive layers of composition 01 having basis weights of 36.5, 63.5, and 106.4 g/m² in comparison to a layer with comparative formulation V1 having a basis weight of 100 g/m².

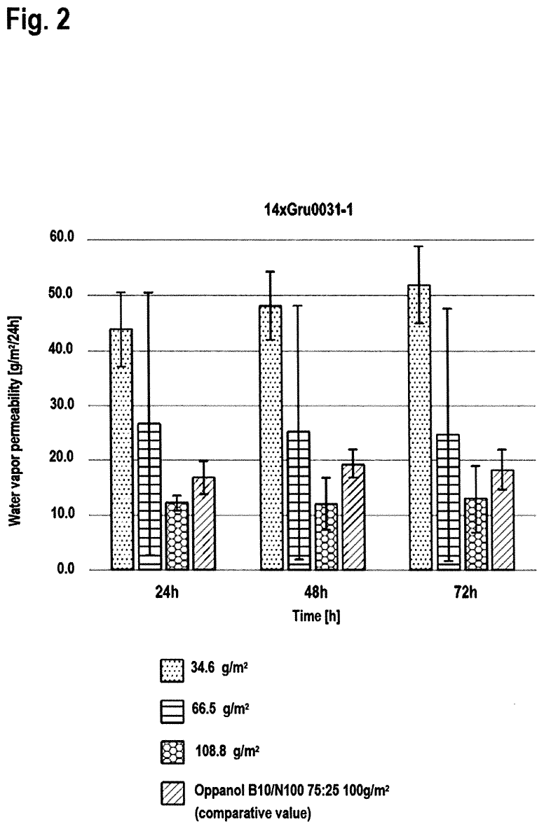

FIG. 2: Water vapor permeability of three occlusive layers of composition 02 having basis weights of 34.6, 66.5, and 108.8 g/m² in comparison to a layer with comparative formulation V2 having a basis weight of 100 g/m².

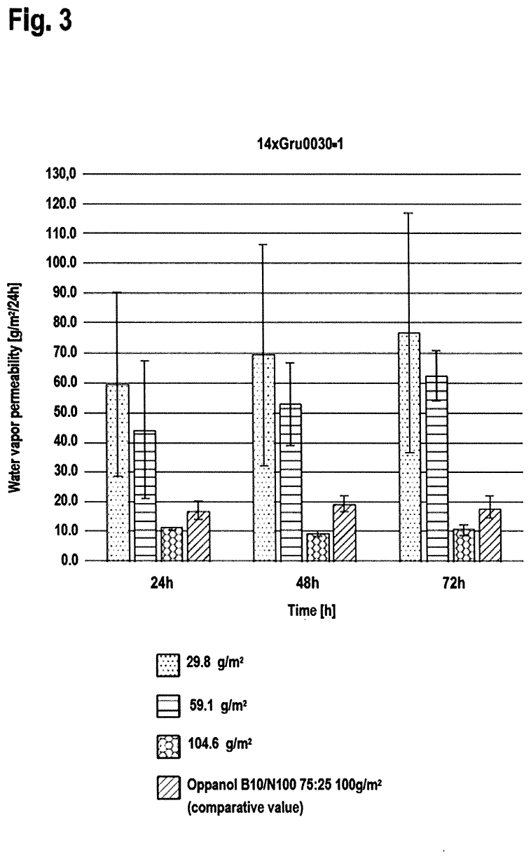

FIG. 3: Water vapor permeability of three occlusive layers of composition 03 having basis weights of 29.8, 59.1, and 104.6 g/m² in comparison to a layer with comparative formulation V2 having a basis weight of 100 g/m².

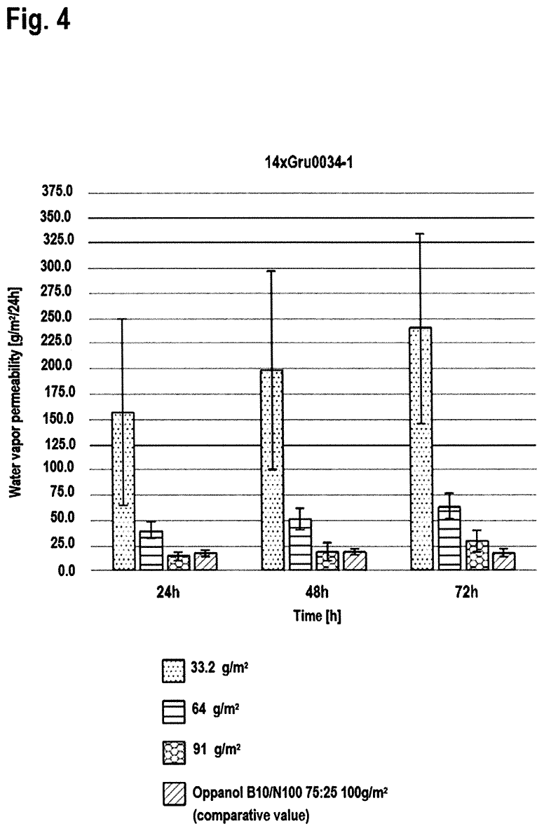

FIG. 4: Water vapor permeability of three occlusive layers of composition 04 having basis weights of 33.2, 64, and 91 g/m² in comparison to a layer with comparative formulation V2 having a basis weight of 100 g/m².

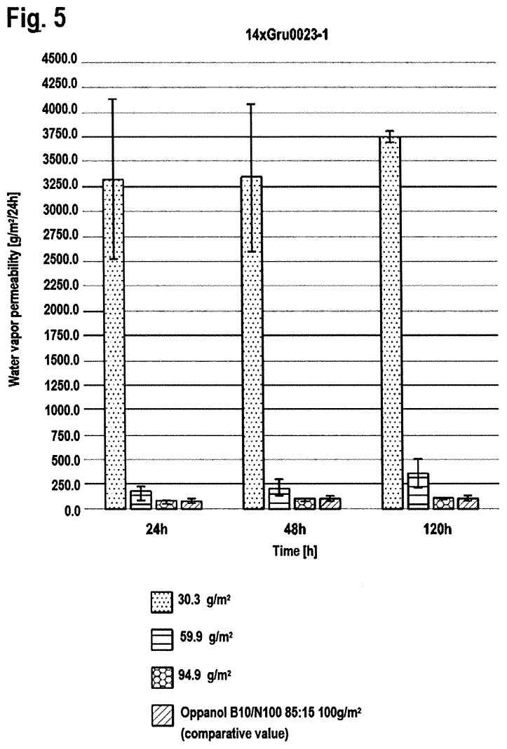

FIG. 5: Water vapor permeability of three occlusive layers of composition 05 having basis weights of 30.3, 59.9, and 94.9 g/m² in comparison to a layer with comparative formulation V1 having a basis weight of 100 g/m².

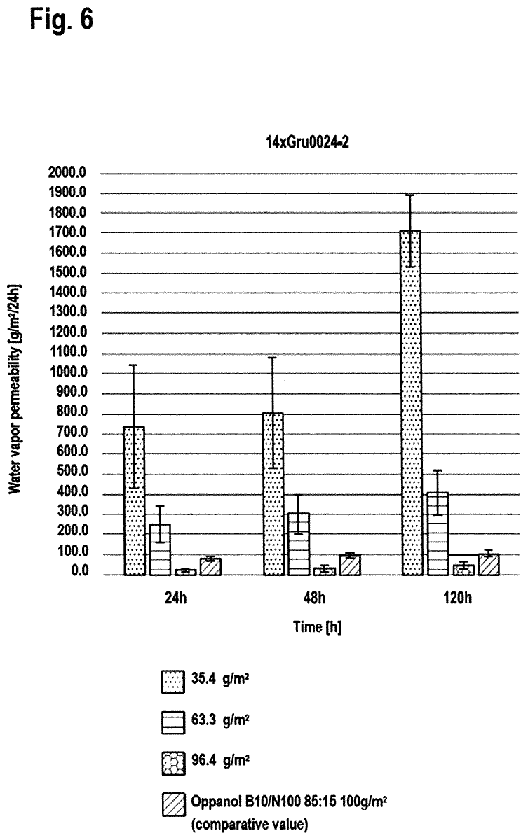

FIG. 6: Water vapor permeability of three occlusive layers of composition 06 having basis weights of 35.4 m, 63.3, and 96.4 g/m² in comparison to a layer with comparative formulation V1 having a basis weight of 100 g/m².

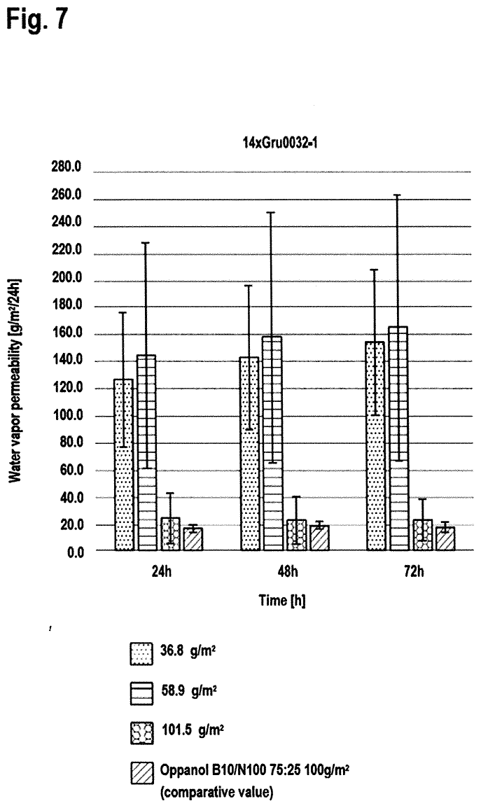

FIG. 7: Water vapor permeability of three occlusive layers of composition 06 having basis weights of 36.8, 58.9, and 101.5 g/m² in comparison to a layer with comparative formulation V2 having a basis weight of 100 g/m².

### Example 2

| | LTS | Composition [%] | | | |
|---|---|---|---|---|---|
| | formulation | Oppanol | Oppanol | SIS | Hydrobrite |
| | code | B10 | N100 | 5229P | HV |
| V1 | 14xGru0016-1 | 85 | 15 | — | — |
| V2 | 14xGru0015-1 | 75 | 25 | — | — |

| | | |
|---|---|---|
| V3 | 14xGru0011-1 | Durotak 387-2287 |

The water vapor permeability of various systems was measured. For this, only an occlusive layer was applied to a flexible, non-occlusive backing layer. The water vapor permeability was determined according to DIN EN 13726-2:2002 at a temperature of 37° C. and a relative humidity of 30%.

The water vapor permeability of an occlusive layer containing medium molecular weight polyisobutylene (Oppanol B10) and high molecular weight polyisobutylene (Oppanol N100) in a ratio of 75:25 (formulation V1) or 85:15 without SIS (formulation V2) was determined in comparison to a layer consisting of DuroTak 387-2287, an acrylate copolymer with free hydroxyl groups (formulation V3). In each case, the basis weight was 100 g/m².

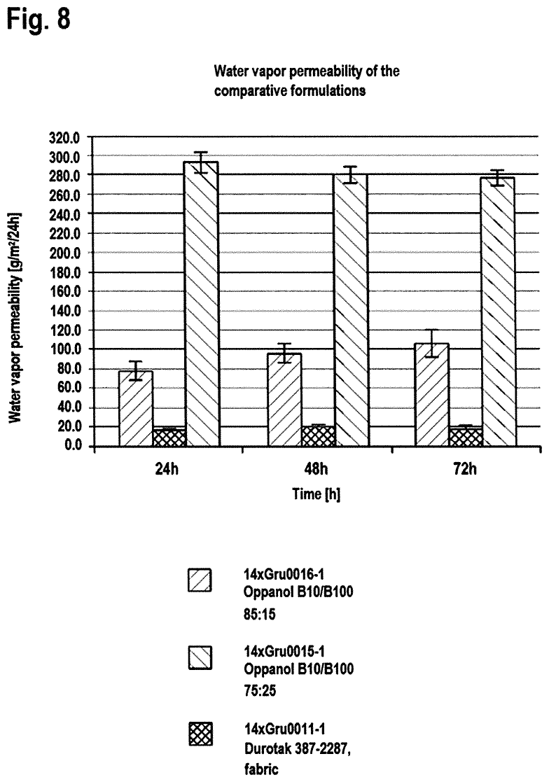

The results are summarized in FIG. 8.

### Example 3

The tackiness of various occlusive layers (backing layer: woven fabric) as described in examples 1 and 2 was investigated on a backing layer and on human skin.

All occlusive layers showed good adhesion and no residue remained on the skin.

### Example 4

Five different transdermal therapeutic systems for the administration of rotigotine were investigated with regard to in vitro human skin penetration.

### Composition of the Active Ingredient-Containing Layer

| | solid | liquid |
|---|---|---|
| | [%] | [%] |
| Rotigotine | 7.50 | 4.665 |
| Polyvinylpyrrolidone | 3.33 | 2.07 |
| (Kollidon 90 F) | | |
| Ethanol | — | 14.04 |
| Sodium metabisulfite | 0.0015 | 0.0093 |
| solution (10 wt %) | | |
| Ascorbyl palmitate | 0.017 | 0.010 |
| DL-α-Tocopherol | 0.042 | 0.026 |
| BIO-PSA Q7-4301 | 53.47 | 47.515 |
| (70.0% w/w) | | |
| BIO-PSA Q7-4201 | 35.64 | 31.67 |
| (70.0% w/w) | | |
| Total | 100.00 | 100.005 |

### Preparation of the Layer Containing the Active Ingredient:

6.66 g polyvinylpyrrolidone (PVP, Kollidon 90F), 0.083 g DL-α-tocopherol, 0.033 g ascorbyl palmitate and 0.030 g of an aqueous sodium metabisulfite solution (10 wt %) are mixed with 25.93 g anhydrous ethanol to obtain a clear solution (300-2000 rpm; propeller stirrer). 15.00 g rotigotine in polymorph form II is added with stirring at 300 rpm and heated to 60° C. for 90 min.

152.80 g silicone adhesive BIO-PSA 7-4301 (70 wt % in n-heptane) and 101.84 g silicone adhesive BIO-PSA 7-4201 (70 wt % in n-heptane) are added to this mixture and stirred for 10 min at 2000 rpm (propeller stirrer) to obtain a stable dispersion.

The resulting mixture is placed on a suitable polyester release liner (e.g. Scotchpak™ 9744). The coated release liners are dried in a drying oven at 50° C. for 30 minutes and then at about 110° C. for 10 minutes. The coating thickness was selected so that the removal of the solvent results in a basis weight of the rotigotine-containing layer of 60 g/m².

### Manufacturing of the Transdermal Systems:

The rotigotine-containing layer is laminated onto a corresponding PIB mixture laminate and provided with a bi-elastic backing layer KOB TAN 053.

Finally, individual TTS with a size of 10 cm² were punched out of the rotigotine-containing, self-adhesive layer structure and sealed in bags.

### Composition of the Transdermal Systems

| | Active | Occlusive layer | | |
|---|---|---|---|---|
| Transdermal | ingredient | | Layer | Backing |
| system | layer | Formulation | thickness | layer |
| 1 (comparison) | Rotigotine | — | — | PET film |
| 2 | Rotigotine | O1 | 106.6 g/m² | Woven |
| 3 | Rotigotine | O3 | 104.6 g/cm² | Woven |
| 4 | Rotigotine | O2 | 66.5 g/cm² | Woven |

The in vitro human skin penetration of the systems listed in Table 3 was measured using a Franz cell. The donor compartment contains the TTS formulation. The acceptor compartment is filled with buffer or other solutions. By frequently taking samples from the acceptor compartment, the penetration of a substance through the skin can be monitored over the selected period of time. The influence of penetration enhancers on the penetration of a substance can also be tested using this system. The use of the Franz cell as a diffusion model is particularly suitable for predicting the transport of drugs through human skin (=penetration), which corresponds to systemic availability. However, it is important to note that there is no in vitro-in vivo correlation. The Franz cell was loaded with human abdominal skin obtained from operations. Here, 500 μm of dermatomized skin with a diffusion area of 1.172 cm² was incubated with the topical therapeutic system. A phosphate buffer+0.1% NaN₃ (pH=5.5) with a filling volume of 10 mL was used as the acceptor medium. The penetration measurement was carried out at a temperature of 32° C.

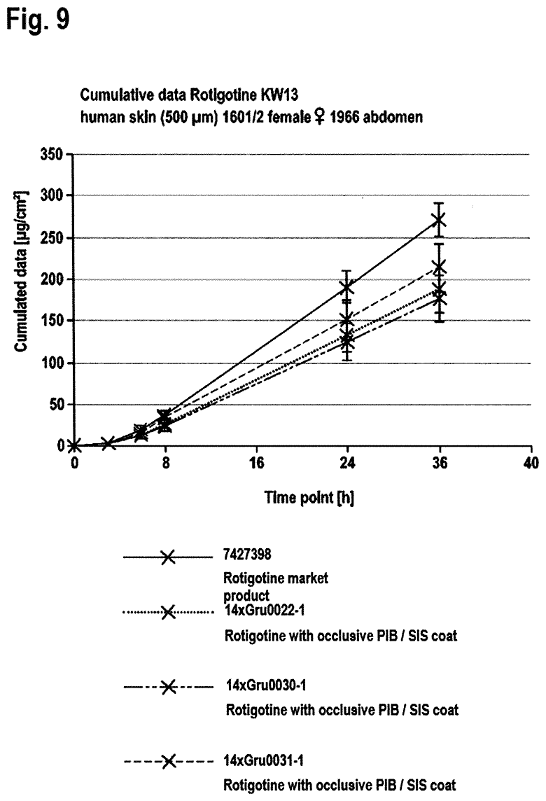

The measurement results are shown in FIG. 9.

1. A transdermal therapeutic system comprising a backing layer, an occlusive layer, and at least one active pharmaceutical ingredient, wherein the occlusive layer comprises at least one occlusive adhesive component based on at least one polyisobutylene and at least one styrene block copolymer, wherein the at least one polyisobutylene is present in an amount of 80 to 97.5 wt % and the one styrene block copolymer is present in an amount of 2.5 to 20 wt %, each based on the total weight of the occlusive layer, in the occlusive layer.

2. The transdermal therapeutic system according to claim 1, characterized in that the at least one polyisobutylene is a mixture comprising a medium molecular weight polyisobutylene having a molecular weight of 20,000 to 60,000 g/mol and a high molecular weight polyisobutylene having a molecular weight of 1,000,000 to 1,200,000 g/mol.

3. The transdermal therapeutic system according to claim 1, characterized in that the at least one styrene block copolymer comprises a styrene-isoprene-styrene block copolymer, a styrene-ethylene-styrene block copolymer, a styrene-butadiene-styrene block copolymer, a styrene-ethylene-butylene-styrene block copolymer, and/or a styrene-ethylene-propylene-styrene block copolymer.

4. The transdermal therapeutic system according to claim 1, characterized in that the at least one polyisobutylene is a mixture comprising a medium molecular weight polyisobutylene and a high molecular weight polyisobutylene in a weight ratio of 95:5 to 75:25.

5. The transdermal therapeutic system according to claim 1, characterized in that the at least one polyisobutylene is contained in the occlusive layer in an amount of 85 to 95 wt %, based on the total weight of the occlusive layer.

6. The transdermal therapeutic system according to claim 1, characterized in that the at least one styrene block copolymer is contained in the occlusive layer in an amount of 5 to 15 wt %, based on the total weight of the occlusive layer.

7. The transdermal therapeutic system according to claim 1, characterized in that the basis weight of the occlusive layer is from 30 to 250 g/m².

8. The transdermal therapeutic system according to claim 1, characterized in that the transdermal therapeutic system additionally comprises a matrix layer, wherein the occlusive layer is arranged between the backing layer and the matrix layer, wherein the matrix layer comprises at least one pressure-sensitive adhesive and the at least one active pharmaceutical ingredient.

9. The transdermal therapeutic system according to claim 8, characterized in that the at least one pressure-sensitive adhesive comprises a pressure-sensitive adhesive based on silicone, based on a (meth)acrylate polymer, and/or based on a (meth)acrylate copolymer.

10. The transdermal therapeutic system according to claim 1, characterized in that the at least one active pharmaceutical ingredient is selected from the group comprising hypnotics, sedatives, antieleptics, amphetamines, psychoneurotropics, neuroleptics, neuromuscular-blocking agents, antispasmodics, antihistamines, antiallergics, cardiotonic agents, antiarrhythmics, diuretics, hypotensive agents, vasopressors, antitussives, expectorants, analgesics, thyroid hormones, sex hormones, glucocorticoid hormones, antidiabetics, antitumor agents, antibiotics, chemotherapeutics, narcotics, anti-Parkinson agents, anti-Alzheimer agents and/or triptans.

11. The transdermal therapeutic system according to claim 1, characterized in that the at least one active pharmaceutical ingredient is present in an amount of 1 to 30 wt % in the occlusive layer, based on the total weight of the occlusive layer, and/or in the matrix layer, based on the total weight of the matrix layer.

12. The transdermal therapeutic system according to claim 1, characterized in that the backing layer comprises an elastic woven, knitted, or non-woven fabric.

13. The transdermal therapeutic system according to claim 1, characterized in that the backing layer has an elasticity of 1 to 100%, in the longitudinal and/or transverse direction, but not in the thickness direction, in relation to the initial state of the material.

14. A medicinal product comprising the transdermal therapeutic system according to claim 1.

15. The transdermal therapeutic system according to claim 1, characterized in that the basis weight of the occlusive layer is from 50 to 200 g/m².

16. The transdermal therapeutic system according to claim 1, characterized in that the backing layer has an elasticity of 10 to 50% in the longitudinal and/or transverse direction, but not in the thickness direction, in relation to the initial state of the material.

17. The transdermal therapeutic system according to claim 1, characterized in that the backing layer has an elasticity of 15 to 30% in the longitudinal and/or transverse direction, but not in the thickness direction, in relation to the initial state of the material.
